(19) United States
(12) Patent Application Publication
(10) Pub. No.: US 2017/0304364 A1
(43) Pub. Date: Oct. 26, 2017

(54) NK-92 CELLS IN COMBINATION THERAPY WITH CANCER DRUGS

(71) Applicant: NantKwest, Inc., Culver City, CA (US)

(72) Inventors: Tien Lee, Culver City, CA (US); Hans G. Klingemann, Culver City, CA (US); Barry J. Simon, Culver City, CA (US)

(73) Assignee: NantKwest, Inc., Culver City, CA (US)

(21) Appl. No.: 15/526,157

(22) PCT Filed: Mar. 25, 2016
(86) PCT No.: PCT/US2016/024360

§ 371 Date: May 11, 2017

Related U.S. Application Data
(60) Provisional application No. 62/139,330, filed on Mar. 27, 2015.

(51) Int. Cl. A61K 35/17; A61K 31/337; A61K 31/454; A61K 45/06

(52) U.S. Cl. CPC A61K 35/17; A61K 45/06; A61K 31/337; A61K 31/454

(57) ABSTRACT

This disclosure is directed to compositions and methods for treating cancer using combination therapies of NK-92 cells with cancer drugs (e.g. thalidomide, cisplatin, and paclitaxel).

### CROSS-REFERENCE TO RELATED PATENT APPLICATIONS

This application claims the benefit of priority to U.S. Provisional Application No. 62/139,330, filed Mar. 27, 2015, which is incorporated by reference herein in its entirety.

### BACKGROUND OF THE INVENTION

Chemotherapy involves the disruption of cell replication or cell metabolism, and it remains one of the main treatment options for cancer. Chemotherapy can be effective, but there are severe side effects, e.g., vomiting, low white blood cells (WBC), loss of hair, loss of weight and other toxic effects. Because of the extremely toxic side effects, many cancer individuals cannot successfully finish a complete chemotherapy regime. Cancer drug monotherapy also selects for mutant cancer cells that are resistant to the drug.

Advances in immunotherapy poses some benefits and involves the use of certain cells of the immune system that have cytotoxic activity against particular target cells. Natural killer (NK) cells are cytotoxic lymphocytes that constitute a major component of the innate immune system. Natural killer (NK) cells, generally representing about 10-15% of circulating lymphocytes, bind and kill targeted cells, including virus-infected cells and many malignant cells, non-specifically with regard to antigen and without prior immune sensitization. Herberman et al., Science 214:24 (1981). Killing of targeted cells occurs by inducing cell lysis. NK cells used for this purpose are isolated from the peripheral blood lymphocyte (“PBL”) fraction of blood from the subject, expanded in cell culture in order to obtain sufficient numbers of cells, and then re-infused into the subject, NK cells have been shown to be somewhat effective in both ex vivo therapy and in vivo treatment. However, such therapy is complicated by the fact that not all NK cells are cytolytic and the therapy is specific to the treated patient.

NK-92 is a cytolytic cancer cell line which was discovered in the blood of a subject suffering from a non-Hodgkins lymphoma and then immortalized ex vivo. NK-92 cells are derived from NK cells, but lack the major inhibitory receptors that are displayed by normal NK cells, while retaining the majority of the activating receptors. NK-92 cells do not, however, attack normal cells nor do they elicit an unacceptable immune rejection response in humans. Characterization of the NK-92 cell line is disclosed in WO 1998/49268 and U.S. Patent Application Publication No. 2002-0068044. NK-92 cells have been evaluated as a therapeutic agent in the treatment of certain cancers. The therapeutic use of NK-92 remains, however, unpredictable.

Due to the severity and breadth of cancer, there is still a great need for effective treatments of such diseases or disorders that overcome the shortcomings of chemotherapy.

### SUMMARY OF THE INVENTION

Described herein are compositions comprising at least one NK-92 cell and at least one cancer drug (e.g., thalidomide, cisplatin, and paclitaxel), use of the compositions for treatment of cancer, and methods of treating a subject having (or suspected of having) cancer by administering the compositions to the subject. The compositions and methods provide the unexpected and surprising result that the combination of NK-92 cells with cancer drugs allows for lower doses of the cancer drugs to be administered than if the cancer drug is administered alone (i.e., without the NK-92 cells), thus, decreasing the harmful side effects of many cancer drugs.

In one aspect, the composition comprises or consists of at least one NK-92 cell and at least one cancer drug. In some embodiments, the composition comprises or consists of a plurality of NK-92 cells and a cancer drug. The plurality of NK-92 cells can include a plurality of identical or substantially identical NK-92 cells; e.g., NK-92 cells derived from a single clone and having identical, substantially identical or similar phenotypes, such as expressing the same surface markers. The term “substantially identical” includes normal and expected variation in the phenotype of clonally related cells. In sonic embodiments, the plurality of NK-92 cells includes a mixture of cells having different phenotypes; e.g., cells derived from different parental clones and/or expressing different surface markers. Further, in some embodiments, the composition comprises NK-92 cells that are modified to express at least one marker on the surface of the cell.

In some embodiments, the cancer drug is selected from the group consisting of: thalidomide, cisplatin (cis-DDP), oxaliplatin, carboplatin, anthracenediones, mitoxantrone; hydroxyurea, methylhydrazine derivatives, procarbazine (N-methylhydrazine, MIH), adrenocortical suppressants, mitotane (o,p′-DDD), aminoglutethimide, RXR agonists, bexarotene, tyrosine kinase inhibitors, imatinib, mechlorethamine, cyclophosphamide, ifosfaniide, melphalan (L-sarcolysin), chlorambucil, ethylenimines, methylmelamines, hexamethylmelamine, thiotepa, busulfan, carmustine (BCNU), semustine (methyl-CCNU), lomustine (CCNU), streptozocin (streptozotocin), DNA synthesis antagonists, estratnustine phosphate, triazines, dacarbazine (DTIC, dimethyl-triazenoimidazolecarboxamide), temozolomide, folic acid analogs, methotrexate (amethopterin), pyrimidine analogs, fluorouracin (5-fluorouracil, 5-FU, 5FU), floxuridine (fluorodeoxvuridine, FUdR), cytarabine (cytosine arabinoside), gemcitabine, purine analogs, mercaptopurine (6-mercaptopurine, 6-MP), thioguanine (6-thioguanine, TG), pentostatin (2′-deoxycoformycin, deoxycoformycin), cladribine and fludarabine, topoisomerase inhibitors, amsacrine, vinca alkaloids, vinblastine (VLB), vincristine, taxanes, paclitaxel, protein bound paclitaxel (Abraxane®), docetaxel (Taxotere®); epipodophyllotoxins, etoposide, teniposide, camptothecins, topotecan, irinotecan, dactinomycin (actinomycin D), daunorubicin (daunomycin, rubidomycin), doxorubicin, bleomycin, mitomycin (mitomycin C), idarubicin, epirubicin, buserelin, adrenocorticosteroids, prednisone, progestins, hydroxyprogesterone caproate, medroxyprogesterone acetate, megestrol acetate, diethylstilbestrol, ethinyl estradiol, tamoxifen, anastrozole; testosterone propionate, fluoxymesterone, flutamide, bicalutamide, and leuprolide.

Thus, in some embodiments, the cancer drug is thalidomide or its derivatives. In some embodiments, the cancer drug is selected from the group consisting of cisplatin, carboplatin, and oxaliplatin. In certain embodiments, the cancer drug is selected from the group consisting of paclitaxel, Abraxane®, and Taxotere®. In one embodiment, the cancer drug is selected from the group consisting of asparaginase, bevacizumab, bleomycin, doxorubicin, epirubicin, etoposide, 5-fluorouracil, hydroxyurea, streptozocin, and 6-mercaptopurine, cyclophosphamide, paclitaxel, and gemcitabine.

In some embodiments, the amount of the cancer drug in the composition is less than the amount of the drug in a composition without at least one NK-92 cell.

In one aspect, methods for treating cancer in a subject in need thereof are described, the method comprising administering to the subject an effective amount of the compositions described herein. In some embodiments, the method comprises administering to the subject an effective amount of natural killer cells and an effective amount of cancer drugs. In some embodiments, the method comprises administering to the subject an effective amount of at least one cancer drug and at least one NK-92 cell.

In some embodiments, the cancer is selected from the group consisting of colorectal cancer, breast cancer, lung cancer, prostate cancer, pancreatic cancer, bladder cancer, cervical cancer, cholangiocarcinoma, gastric sarcoma, glioma, leukemia, lymphoma, melanoma, multiple myeloma, osteosarcoma, ovarian cancer, stomach cancer, brain cancer.

In some embodiments, the cancer drug is thalidomide or its derivatives. In one embodiment, the cancer drug is selected from the group consisting of cisplatin, carboplatin, and oxaliplatin. In some embodiments, the cancer drug is selected from the group consisting of paclitaxel, Abraxan, and Taxotere. In certain embodiments, the cancer drug is selected from the group consisting of asparaginase, bevacizumab, bleomycin, doxorubicin, epirubicin, etoposide, 5-fluorouracil, hydroxyurea, streptozocin, and 6-mercaptopurine, cyclophosphamide, paclitaxel, and gemcitabine.

In some embodiments, the subject is selected from the group consisting of bovines, swine, rabbits, alpacas, horses, canines, felines, ferrets, rats, mice, fowl and buffalo. In one embodiment, the subject is human.

The cancer drug and the NK-92. cells can be administered simultaneously or sequentially. In some embodiments, the cancer drug and the NK-92 cells are mixed together prior to administering to the subject. In certain embodiments, the cancer drug is administered before the administration of the NK-92 cells, and the NK-92 cells are administered after the cancer drug is removed from the subject.

In some embodiments, the effective amount of the cancer drug administered to the subject is less than the effective or optimal amount of the drug administered alone (i.e., without at least one NK-92 cell). For example, in some embodiments, the cancer drug is paclitaxel, and the dose of paclitaxel in combination with NK-92 cells administered to the subject is less than the standard or optimal dose of paclitaxel administered alone. In some embodiments, the lower dose of cancer drug in combination with NK-92 cells results in a significant decrease in tumor burden compared to administering the cancer drug and NK-92 cells separately.

In some embodiments of the method, the NK92 cell is modified to express at least one marker or a chimeric antigen receptor on the surface of the cell.

In some embodiments, the combination of at least one cancer drug and at least one NK-92 cell provides a synergistic result compared to the administration of the cancer drug or NK-92 cell alone. In one embodiment, the cancer drug is paclitaxel, and the cancer is breast cancer.

In another aspect, the disclosure provides for the use of the compositions described herein for the treatment of cancers or tumors. In some embodiments, provided herein are compositions for use in the preparation of a medicament for the treatment of cancers or tumors. Thus, in some embodiments, described herein is a composition comprising at least one NK-92 cell and at least one cancer drug for use in the treatment of cancers or tumors. In some embodiments, described herein is use of at least one NK-92 cell and at least one cancer drug in the preparation of a medicament for the treatment of cancers or tumors.

### BRIEF DESCRIPTION OF THE DRAWINGS

FIG. 1 shows the dosing schedule described in Example 1.

FIG. 2 shows the post-treatment change in tumor volume described in Example 1.

FIG. 3 shows the post-treatment change in body weight described in Example 1.

### DETAILED DESCRIPTION OF TIIE INVENTION

Described herein are compositions comprising a combination of NK-92 cells and one or more cancer drugs. The compositions are useful for treating cancer or for preparing medicaments for treating cancer. Also described are methods of treating a subject having cancer by administering a composition described herein. The methods provide the unexpected and surprising result that administering a cancer drug in combination with NK-92 cells allows for lower doses of the cancer drug to be administered compared to the typical “standard of care” dose administered by a physician if the cancer drug is administered alone (without the NK-92 cells), thereby decreasing the harmful side effects andior the cost of many cancer drugs. Thus, the disclosure describes that co-administration of both NK-92 cells and a cancer drug provides a synergistic effect, such that the treatment is more effective than the additive effect when either the NK-92 cells or the cancer drug is administered alone.

After reading this description, it will become apparent to one skilled in the art of cancer immunotherapy how to implement various alternative embodiments and alternative applications of those described herein. However, not all embodiments are described herein. It will be understood that the embodiments presented here are presented by way of an example only, and not limitation. As such, this detailed description of various alternative embodiments should not be construed to limit the scope or breadth of the present disclosure or claims as set forth below.

It is understood that the aspects described below are not limited to specific compositions, methods of preparing such compositions, or uses thereof as such may, of course, vary. It is also understood that the terminology used herein is for the purpose of describing particular aspects only and is not intended to be limiting.

### Definitions

Unless defined otherwise, all technical and scientific terms used herein have the same meaning as commonly understood by one of ordinary skill in the art in the field of cancer immunotherapy.

In this specification and in the claims that follow, reference will be made to a number of terms that shall be defined to have the following meanings:

The terminology used herein is for the purpose of describing particular embodiments only and is not intended to be limiting. As used herein, the singular forms “a”, “an” and “the” are intended to include the plural forms as well, unless the context clearly indicates otherwise.

All numerical designations, e.g., pH, temperature, time, concentration, amounts, and molecular weight, including ranges, are approximations which are varied (+) or (−) by increments of 0.1 or 1.0, where appropriate. It is to be understood, although not always explicitly stated, that all numerical designations may be preceded by the term “about.” The term “about” includes variations that are normally encountered by one of ordinary skill in the art in the field of cancer immunotherapy. For example, the term about includes (+) or (−) 0.1, 0.5, 1.0, 2.0. 3.0, 4.0, 5.0, 6.0, 7.0, 8.0, 9.0, or 10.0% of a recited numerical value or range. It is also to be understood, although not always explicitly stated, that the reagents described herein are merely exemplary and that equivalents of such are known in the art.

“Optional” or “optionally” means that the subsequently described event or circumstance can or cannot occur, and that the description includes instances where the event or circumstance occurs and instances where it does not.

The term “comprising” or “comprises” is intended to mean that the compositions and methods include the recited elements, but do not exclude others. “Consisting essentially of” when used to define compositions and methods, shall mean excluding other elements and steps that materially affect the basic and novel characteristic(s) of the claimed invention. For example, a composition consisting essentially of the elements as defined herein would not exclude other elements that do not materially affect the basic and novel characteristic(s) of the claimed invention. “Consisting of” shall mean excluding more than trace amounts of other ingredients and substantial method steps recited. Embodiments defined by each of these transition terms are within the scope of the claims.

As used herein, the term “cancer drugs” refers to conventional and well known chemical and biological (i.e, non-cellular) agents used to treat cancer and is sometimes referred to as “conventional therapy” or “conventional treatment”. Such conventional therapy includes, but is not limited to, chemotherapy using anti-tumor chemicals, radiation therapy, hormonal therapy, and the like as well as combinations thereof. The term can also include antibodies and fragments thereof that are useful to treat or prevent cancer or tumors.

The terms “patient,” “subject,” “individual,” and the like are used interchangeably herein, and refer to any animal, or cells thereof whether in vitro or in situ, amenable to the methods described herein. In certain non-limiting embodi merits, the patient, subject or individual is a human.

The term “treating” or “treatment” covers the treatment of a disease or disorder described herein, in a subject, such as a human, and includes: (i) inhibiting a disease or disorder, i.e., arresting its development; (ii) relieving a disease or disorder, i.e., causing regression of the disorder (iii) slowing progression of the disorder and/or (iv) inhibiting, relieving, or slowing progression of one or more symptoms of the disease or disorder. The term “administering” or “administration” of an agent, drug, or a natural killer cell to a subject includes any route of introducing or delivering to a subject a compound to perform its intended function. Administration can be carried out by any suitable route, including orally, intranasally, parenterally (intravenously, intramuscularly, intraperitoneally, or subcutaneously), or topically. Administration includes self-administration and the administration by another.

It is also to be appreciated that the various modes of treatment or prevention of medical diseases and conditions as described are intended to mean “substantial,” which includes total but also less than total treatment or prevention, and wherein some biologically or medically relevant result is achieved. The treatment may be a continuous prolonged treatment for a chronic disease or a single, or few time administrations for the treatment of an acute condition.

The term “separate” administration refers to an administration of at least two active ingredients at the same time or substantially the same time by different routes.

The term “sequential” administration refers to administration of at least two active ingredients at different times, the administration route being identical or different. More particularly, sequential use refers to the whole administration of one of the active ingredients before administration of the other or others commences. It is thus possible to administer one of the active ingredients over several minutes, hours, or days before administering the other active ingredient or ingredients. The term “sequential” therefore is different than “simultaneous” administration.

The term “simultaneous” administration refers to the administration of at least two active ingredients by the same route at the same time or at substantially the same time.

The term “therapeutic” as used herein means a treatment and/or prophylaxis. A therapeutic effect is obtained by suppression, remission, or eradication of a disease state.

The term “therapeutically effective amount” refers to an amount of a therapeutic agent that (e.g., an anti-cancer or anti-tumor agent), when administered to a subject, is sufficient to treat a disease or disorder (e.g., a solid mass tumor or other type of cancer). The therapeutically effective amount of the anti-tumor agent will vary depending on the tumor being treated and its severity as well as the age, weight, etc., of the patient to be treated. The skilled artisan will be able to determine appropriate dosages depending on these and other factors. The compositions can also be administered in combination with one or more additional therapeutic compounds. In the methods described herein, the therapeutic compounds may be administered to a subject having one or more signs or symptoms of a disease or disorder.

As used herein, “immunotherapy” refers to the use of NK-92 cells, modified or unmodified, in combination with antibody, naturally occurring or modified NK cell or T-cell, whether alone or in combination, and which are capable of inducing cytotoxicity when contacting a target cell.

As used herein, “natural killer (NK) cells” are cells of the immune system that kill target cells in the absence of a specific antigenic stimulus, and without restriction according to MHC class. Target cells may be tumor cells or cells harboring viruses. NK cells are characterized by the presence of CD56 and the absence of CD3 surface markers.

The term “endogenous NK cells” is used to refer to NK cells derived from a donor (or the patient), as distinguished from the NK-92 cell line. Endogenous NK cells are generally heterogeneous populations of cells within which NK cells have been enriched. Endogenous NK cells may be intended for autologous or allogeneic treatment of a patient.

“NK-92 cells” refer to the immortal NK cell line, NK-92, which was originally obtained from a patient having non-Hodgkin's lymphoma. For purposes of this disclosure and unless indicated otherwise, the term “NK-92” is intended to refer to the original NK-92 cell lines as well as NK-92 cell lines that have been modified (e.g., by introduction of exogenous genes). NK-92 cells and exemplary and non-limiting modifications thereof are described in U.S. Pat. Nos. 7,618,817; 8,034,332; and 8,313,943, all of which are incorporated herein by reference in their entireties.

As used herein, “non-irradiated NK-92 cells” are NK-92 cells that have not been irradiated. Irradiation renders the cells incapable of growth and proliferation. It is envisioned that the NK-92 cells will be irradiated at the treatment facility or some other point prior to treatment of a subject, since the time between irradiation and infusion should be no longer than four hours in order to preserve optimal activity. Alternatively, NK-92 cells may be inactivated by another mechanism.

As used herein, “inactivation” of the NK-92 cells renders them incapable of growth. Inactivation may also relate to the death of the NK-92 cells. In some embodiments, NK-92 cells are inactivated after they have effectively purged an ex vivo sample of cells related to a pathology in a therapeutic application, or after they have resided within the body of a mammal a sufficient period of time to effectively kill many or all target cells residing within the body. Inactivation can be induced, by way of non-limiting example, by administering an inactivating agent to which the NK-92 cells are sensitive.

“Modified NK-92 cell” refers to an NK-92 cell that is genetically modified to express at least one cell marker, or further comprises a vector that encodes for transgenes, including but not limited to CD16, chimeric antigen receptor, IL-2, and/or suicide genes.

As used herein. “non-irradiated NK-92 cells” are NK-92 cells that have not been irradiated. Irradiation renders the cells incapable of growth and proliferation. In some embodiments, the NK-92 cells will be irradiated at the treatment facility or some other point prior to treatment of a patient, since the time between irradiation and infusion should be no longer than four hours in order to preserve optimal activity. Alternatively, NK-92. cells can be inactivated by another mechanism.

As used herein, the terms “cytotoxic” and “cytolytic”, when used to describe the activity of effector cells such as NK cells, are intended to be synonymous. In general, cytotoxic activity relates to killing of target cells by any of a variety of biological, biochemical, or biophysical mechanisms. Cytolysis refers more specifically to activity in which the effector lyses the plasma membrane of the target cell, thereby destroying its physical integrity. This results in the killing of the target cell. Without wishing to be bound by theory, it is believed that the cytotoxic effect of NK cells is due to cytolysis.

The term “kill” with respect to a cell/cell population includes any type of manipulation that will lead to the death of that cell/cell population.

The term “Fc receptor” refers to a protein found on the surface of certain cells (e.g., natural killer cells) that contributes to the protective functions of immune cells by binding to part of an antibody known as the Fc region. Binding of the Fc region to the Fc receptor (FCR) of a cell stimulates phagocytic or cytotoxic activity of a cell via antibody-mediated phagocytosis or antibody-dependent cell-mediated cytotoxicity (ADCC). FcRs are classified based on the type of antibody they recognize. For example, Fc-gamma receptors (FCyR) bind to the IgG class of antibodies. FCγRIII-A (also called CD16) is a low affinity Fc receptor that binds to IgG antibodies and activates ADCC. FCγRIII-A are typically found on NK cells.

The terms “polynucleotide”, “nucleic acid,” and “oligonucleotide” are used interchangeably and refer to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any embodiment described herein that is a polynucleotide encompasses both the double-stranded form and each of two complementary single-stranded forms known or predicted to make up the double-stranded form.

A polynucleotide is composed of a specific sequence of four nucleotide bases: adenine (A); cytosine (C); guanine (G); thymine (T); and uracil (U) for thymine when the polynucleotide is RNA. Thus, the term “polynucleotide sequence” is the alphabetical representation of a polynucleotide molecule.

The terms “identity,” “percent identity,” or “similarity” refer to sequence similarity between amino acid or nucleic acid sequences. Identity or similarity can be determined by comparing a position in each sequence which may be aligned for purposes of comparison. When a position in the compared sequence is occupied by the same base or amino acid, then the molecules are identical at that position. A degree of identity between sequences is a function of the number of matching positions shared by the sequences. An “unrelated” sequence shares less than 40% identity, or alternatively less than 25% identity, with one of the sequences described herein.

The term “express” refers to the production of a gene product, typically RNA or protein, by a cell (in vivo) or in vitro. The term “transient” when referred to expression means a polynucleotide is not incorporated into the genome of the cell.

The terms “polypeptide,” “peptide,” and “protein” are used interchangeably herein to mean a polymer comprising two or more amino acids joined to each other by peptide bonds or modified peptide bonds, i.e., peptide isosteres. Polypeptide refers to both short chains, commonly referred to as peptides, glycopeptides or oligomers, and to longer chains, generally referred to as proteins. Polypeptides may contain amino acids other than the 20 gene-encoded amino acids. Polypeptides include amino acid sequences modified either by natural processes, such as post-translational processing, or by chemical modification techniques that are well known in the art.

The term “cytokine” or “cytokines” refers to the general class of biological molecules which effect cells of the immune system. Exemplary cytokines include but are not limited to interferons and interleukins (IL)—in particular IL-2, IL-12, IL-15, IL-18 and IL-21. In preferred embodiments, the cytokine is IL-2.

As used herein, the term “vector” refers to a non-chromosomal nucleic acid comprising an intact replicon such that the vector may be replicated when placed within a permissive cell, for example by a process of transformation. A vector may replicate in one cell type, such as bacteria, but have limited ability to replicate in another cell, such as mammalian cells. Vectors may be viral or non-viral. Exemplary non-viral vectors for delivering nucleic acid include naked DNA; DNA complexed with cationic lipids, alone or in combination with cationic polymers; anionic and cationic liposomes; DNA-protein complexes and particles comprising DNA condensed with cationic polymers such as heterogeneous polylysine, defined-length oligopeptides, and polyethylene imine, in some cases contained in liposomes; and the use of ternary complexes comprising a virus and polylysine-DNA.

As used herein, the term “targeted” is intended to include, but is not limited to, directing proteins or polypeptides to appropriate destinations in the cell or outside of it. The targeting is typically achieved through signal peptides or targeting peptides, which are a stretch of amino acid residues in a polypeptide chain. These signal peptides can be located anywhere within a polypeptide sequence, but are often located on the N-terminus. Polypeptides can also be engineered to have a signal peptide on the C-terminus. Signal peptides can direct a polypeptide for extracellular section, location to plasma membrane, golgi, endosomes, endoplasmic reticulum, and other cellular compartments. For example, polypeptides with a particular amino acid sequence on their C-terminus (e.g., KDEL) are retained in the ER lumen or transported back the ER lumen.

The terms “synergy” and “synergistic” or are used interchangeably and refer to the interaction or cooperation of two or more substances or agents, such as a cancer drug and an NK-92 cell, to produce a combined effect greater than the sum of their separate effects. Synergistic drug interactions can be determined using the median effect principle (see, Chou and Talalay (1984) Adv Enzyme Regul 22:27 and Synergism and Antagonism in Chemotherapy, Chou and Rideout, eds., 1996, Academic, pp. 61-102) and quantitatively determined by combination indices using the computer program Calcusyn (Chou and Hayball, 1996, Biosoft, Cambridge, Mass.). See also, Reynolds and Maurer, Chapter 14 in Methods in Molecular in Medicine, vol. 110: Chemosensitivity, Vol. 1: In vitro Assays, Blumenthal, ed., 2005, Humana Press. Combination indices (CI) quantify synergy, summation and antagonism as follows: CI<1 (synergy); CI=1 (summation); CI>1 (antagonism). A CI value of 0.7-0.9 indicates moderate to slight synergism. A CI value of 0.3-0.7 indicates synergism. A CI value of 0.1-0.3 indicates strong synergism. A CI value of <0.1 indicates very strong synergism.

Titles or subtitles may be used in the specification for the convenience of a reader, which are not intended to influence the scope of the claims. Additionally, some terms used in this specification are more specifically defined below.

### NK-92 Cells

The NK-92 cell line is a unique cell line that was discovered to proliferate in the presence of interleukin 2 (IL-2). Gong et at., Leukemia 8:652-658 (1994). These cells have high cytolytic activity against a variety of cancers. The NK-92 cell line is a homogeneous cancerous NK cell population having broad anti-tumor cytotoxicity with predictable yield after expansion. Phase I clinical trials have confirmed its safety profile.

The NK-92 cell line is found to exhibit the CD56^{bright}, CD2, CD7, CD28, CD45, and CD54 surface markers. It furthermore does not display the CD1, CD3, CD4,CD5, CD8, CD10, CD14, CD16, CD19, CD20, CD23, and CD34 markers. Growth of NK-92 cells in culture is dependent upon the presence of recombinant interleukin 2 (rIL-2), with a dose as low as 1 IU/mL being sufficient to maintain proliferation. IL-7 and IL-12 do not support long-term growth, nor do other cytokines tested, including IL-1α, IL-6, tumor necrosis factor α, interferon α, and interferon γ, NK-92 has high cytotoxicity even at a low effector:target (E:T) ratio of 1:1. Gong, et at., supra. NK-92 cells are deposited with the American Type Culture Collection (ATCC), designation CRL-2407.

Heretofore, studies on endogenous NK cells have indicated that IL-2 (1000 IU/mL) is critical for NK cell activation during shipment, but that the cells need not be maintained at 37° C. and 5% carbon dioxide. Koepsell, et al., Transfusion 53:398-403 (2013). However, endogenous NK cells are significantly different from NK-92 cells, in large part because of their distinct origins: NK-92 is a cancer-derived cell line, whereas endogenous NK cells are harvested from a donor (or the patient) and processed for infusion into a patient. Endogenous NK cell preparations are heterogeneous cell populations, whereas NK-92 cells are a homogeneous, clonal cell line. NK-92 cells readily proliferate in culture while maintaining cytotoxicity, whereas endogenous NK cells do not. In addition, an endogenous heterogeneous population of NK cells does not aggregate at high density.

In various embodiments, the NK-92 cells administered to a subject include wild-type original NK-92 cells as described herein, as well as genetically modified NK-92 cells, such as original NK-92 cells modified to express CD1.6 or variants thereof, or any marker disclosed herein. In some embodiments, the NK-92 cells are genetically modified to express an exogenous marker, i.e., a marker that is not expressed by the wild-type original NK-92 cells. In some embodiments, the NK-92 cells are genetically modified to express a chimeric antigen receptor (CAR) that binds to an antigen on a cancer or tumor cell. In some embodiments, the NK-92 cell expresses a CAR that binds to the ErbB2 (HER2) antigen. Exemplary NK-92 cells include, but are not limited to, NK-92 cell lines available from American Type Culture Collection (ATCC) under Accession Accession Nos.: PTA 6670, PTA 6672, PTA 8836, PTA 8837, CRL-2407 and CRL-2408.

NK-92 cells can be administered to an individual by absolute numbers of cells, e.g., said individual can be administered from about 1000 cells/injection to up to about 10 billion cells/injection, such as about, at least about, or at most about, 1×10⁸, 1×10⁷, 5×10⁷, 1×10⁶, 5×10⁶, 1×10⁵, 5×10⁵, 1×10⁴, 5×10⁴, 1×10³, 5×10³ (and so forth) NK-92 cells per injection, or any ranges between any two of the numbers, end points inclusive. in other embodiments, NK-92 cells can be administered to such an individual by relative numbers of cells, e.g., said individual can be administered about 1000 cells to up to about 10 billion cells per kilogram of the individual, such as about, at least about, or at most about, 1×10⁸, 1×10⁷, 5×10⁷, 1×10⁶, 5×10⁶, 5×10⁵, 1×10⁴, 5×10⁴, 1×10³, 5×10³ (and so forth) NK-92 cells per kilogram of the individual, or any ranges between any two of the numbers, end points inclusive. in other embodiments, the total dose may calculated by m² of body surface area, including 1×10¹¹, 1×10¹⁰, 1×10⁹, 1×10⁸, or 1×10⁷ cells per m². The average body surface area of a human is 1.6-1.8 m².

The modified NK-92 cells, and optionally an amount of cancer drugs, can be administered once to a subject having cancer or can be administered multiple times, e.g., once every 1, 2, 3, 4, 5, 6, 7, 8, 9, 10, 11, 12, 13, 14, 15, 16, 17, 18, 19, 20, 21, 22 or 23 hours, or once every 1, 2, 3, 4, 5, 6 or 7 days, or once every 1, 2, 3, 4, 5, 6, 7, 8, 9, 10 or more weeks during therapy, or any ranges between any two of the numbers, end points inclusive.

### Cancer Drugs

In one aspect, the disclosure provides methods for treating cancer comprising administering to the subject in need thereof of an effective amount of natural killer cells and an effective amount of one or more cancer drugs. In certain embodiments, the cancer drug refers to a medicament that may be used to treat cancer, and generally has the ability to kill cancerous cells directly. Examples of cancer drugs include, but are not limited to: thalidomide; platinum coordination complexes such as cisplatin (cis-DDP), oxaliplatin and carboplatin; anthracenediones such as mitoxantrone; substituted ureas such as hydroxyurea; methylhydrazine derivatives such as procarbazine (N-methylhydrazine, MIH); adrenocortical suppressants such as mitotane (o,p′-DDD) and aminoglutethimide; RXR agonists such as bexarotene; and tyrosine kinase inhibitors such as sunitimib and imatinib. Examples of additional cancer drugs include alkylating agents, antimetabolites, natural products, hormones and antagonists, and miscellaneous agents. Alternate names are indicated in parentheses. Examples of alkylating agents include nitrogen mustards such as mechlorethamine, cyclophosphainide, ifosfamide, melphalan sarcolysin) and chlorambucil; ethylenimines and methylmelamines such as hexamethylmelamine and thiotepa; alkyl sulfonates such as busulfan; nitrosoureas such as carmustine (BCNU), semustine (methyl-CCNU), lomustine (CCNU) and streptozocin (streptozotocin); DNA synthesis antagonists such as estramustine phosphate; and triazines such as dacarbazine (DTIC, dimethyl-triazenoimidazolecarboxamide) and temozolomide. Examples of antimetabolites include folic acid analogs such as methotrexate (amethopterin); pyrimidine analogs such as fluorouracin (5-fluorouracil, 5-FU, SFU), floxuridine (fluorodeoxyuridine, FUdR), cytarabine (cytosine arabinoside) and gemcitabine; purine analogs such as mercaptopurine (6-mercaptopurine, 6-MP), thioguanine (6-thioguanine, TG) and pentostatin (2′-deoxycoformycin, deoxycoformycin), cladribine and fludarabine; and topoisomerase inhibitors such as amsacrine. Examples of natural products include vinca alkaloids such as vinblastine (VLB) and vincristine; taxanes such as paclitaxel, protein bound paclitaxel (Abraxane) and docetaxel (Taxotere); epipodophyllotoxins such as etoposide and teniposide; camptothecins such as topotecan and irinotecan; antibiotics such as dactinomycin (actinomycin D), daunorubicin (daunomycin, rubidomycin), doxorubicin, bleomycin, mitomycin (mitomycin C), idarubicin, epirubicin; enzymes such as L-asparaginase; and biological response modifiers such as interferon alpha and interlelukin 2. Examples of hormones and antagonists include luteinising releasing hormone agonists such as buserelin; adrenocorticosteroids such as prednisone and related preparations; progestins such as hydroxyprogesterone caproate, rnedroxyprogesterone acetate and megestrol acetate; estrogens such as diethylstilbestrol and ethinyl estradiol and related preparations; estrogen antagonists such as tamoxifen and anastrozole; androgens such as testosterone propionate and fluoxymesterone and related preparations; androgen antagonists such as flutamide and bicalutamide; and gonadotropin-releasing hormone analogs such as leuprolide. Alternate names and trade-names of these and additional examples of cancer drugs, and their methods of use including dosing and administration regimens, will be known to a person versed in the art.

In one aspect, the cancer drug is thalidomide, or a derivative thereof. Thalidomide is a racemic compound sold under the trade name THALOMID© and chemically named α-(N-phthalimido)glutarimide or 2-(2,6-dioxo-3-piperisoindole-1,3(2H)-dione. Thalidomide was originally developed in the 1950's to treat morning sickness, but due to its tetragenic effects was withdrawn from use. Thalidomide is now indicated in the United States for the acute treatment of the cutaneous manifestations of erythema nodosum leprosum. Physicians' Desk Reference, 911-916 (54th ed, 2000). Because its administration to pregnant women can cause birth defects, the sale of thalidomide is strictly controlled. Id.

In addition to treating symptoms of leprosy, thalidomide has reportedly been used to treat chronic graft-vs-host disease, rheumatoid arthritis, sarcoidosis, several inflammatory skin diseases, and inflammatory bowel disease. See generally, Koch, H. P., Prog. Med. Chem. 22:165-242 (1985). See also, Moller, D. R., et al., J. Immunol. 159:5157-5161 (1997); Vasiliauskas, E.A-, et al., Gastroenterology 117:1278-1287 (1999); and Ehrenpreis, E. D., et al., Gastroenterology 117:1271-1277 (1999). It has further been alleged that thalidomide can be combined with other drugs to treat iscehemia/reperfusion associated with coronary and cerebral occlusion. See U.S. Pat. No. 5,643,915, which is incorporated herein by reference.

Thalidomide has reportedly been clinically investigated in the treatment of specific types of cancers. These include refractory multiple myeloma, brain, melanoma, breast, colon, mesothelioma, and renal cell carcinoma. See, e.g., Singhal, S, et al., New England J. Med. 341(21):1565-1571 (1999); and Marx, G. M., et al., Proc. Am. Soc. Clin. Oncology 18:454a (1999). It has further been reported that thalidomide can be used to prevent the development of chronic cardiomyopathy in rats caused by doxorubicin—Costa, P. T., et al., Blood 92(10: suppl.1):235b (1998)—Other reports concerning the use of thalidomide in the treatment of specific cancers include its combination with carboplatin in the treatment of glioblastoma multiforme, McCann, J., Drug Topics 41-42 (Jun. 21, 1999). Thalidomide has reportedly also been used as an antiemetic during the treatment of astrocytoma. Zwart, D-, Arzneim-Forsch 16(12):1688-1689 (1966).

It has been reported that thalidomide is an antiangiogenic agent that can suppress tumor necrosis factor α (INF-α) and Interleukin 12 (IL-12) production. See., e.g., Moller, D. R, et al., J Immunol. 159:5157-5161 (1997); Morena, A. L., et al, J. Exp. Med. 177-1675-16ELO (1993); U.S. Pat. Nos. 5,593,990, 5,629,327, and 5,712,291 to D'Amato and U.S. Pat. No. 5,385,901 to Kaplan. In vitro studies suggest that thalidomide affects the production of a variety of other proteins. See, e.g., McHugh, S. M, et al, Clin. Exp. Immunol. 99:160-167 (1995) Thalidomide may also affect mechanisms related to epithelial or endothelial function or growth. D'amato M., et al., Proc. Natl. Acad. Sci. 91:4082-4085(1994).

In one aspect, the cancer drug is a platinum based drug, which is capable of forming DNA adducts that block DNA and RNA synthesis in cancer cells and inducing apoptosis. Cisplatin (Cis-PtCl₂(NH₃)₂) was approved by the FDA in 1978 for treatment of a variety of cancers and has been used since then for cancer treatment. Cisplatin is given to patients intravenously in saline (sodium chloride solution) and enters the cells by either passive diffusion or other facilitated transport mechanisms. Once inside the cytoplasm, cisplatin undergoes hydrolysis. The chloride ligands are each replaced by a molecule of water, producing a positively charged molecule. Uncharged species are unreactive, but monovalent cations and the divalent cationic species are most reactive.

Cisplatin is a particularly toxic drug. It's severe toxicity includes nephrotoxicity, neurotoxicity and emetogenesis, which is the main dose-limiting factor. It is desirable to develop a formulation which will increase the concentration of cisplatin locally at the tumor site. It is also desirable to reduce the accumulation of cisplatin in other tissues to minimize the toxic side effects.

In one aspect, the cancer drug is paclitaxel, or its derivatives. Paclitaxel is a natural product with antitumor activity. Paclitaxel is obtained via a semisynthetic process from taxus brevifolia and/or taxus baccata. The chemical name for paclitaxel is 5═,20-Epoxy-1,2α,4,7β,10β,13α-hexahydroxytax-11-en-9-one 4, 10-diacetate 2-benzoate 13-ester with (2R,3S)-N-benzoyl-3-phenylisoserine. Paclitaxel is available in the United States of America as TAXOL Injection. Paclitaxel is indicated as first-line and subsequent therapy for the treatment of advanced carcinoma of the ovary. As first-line therapy, paclitaxel is indicated in combination with cisplatin. Paclitaxel is also indicated for the adjuvant treatment of node-positive breast cancer administered sequentially to standard doxorubicin-containing combination chemotherapy. Paclitaxel is also indicated for the treatment of breast cancer after failure of combination chemotherapy for metastatic disease or relapse within 6 months of adjuvant chemotherapy. Paclitaxel, in combination with cisplatin, is also indicated for the first-line treatment of non-small cell lung cancer in patients who are not candidates for potentially curative surgery and/or radiation therapy. Paclitaxel is also indicated for the second-line treatment of AIDS-related Kaposi's sarcoma.

In another aspect, the cancer drug is Abraxane®, a paclitaxel albumin-stabilized nanoparticle formulation, available from Celgene Corp. It is contemplated that other derivatives or formulations of paclitaxel can be used in combination with natural killer cells for the treatment of cancer.

In another aspect, the cancer drug is selected from asparaginase, bevacizumab, bleomycin, doxorubicin, epirubicin, etoposide, 5-fluorouracil, hydroxyurea, streptozocin, and 6-mercaptopurine.

In another aspect, the cancer drug is protease inhibitor such as bortezomib (marketed as Velcade®). In some embodiments, the cancer drug is a receptor protein-tyrosine kinase inhibitor such as imatinib (marketed as Gleevec®) or sunitinib (trade name Sutent®).

In one aspect, the cancer drugs permit efficient natural killer cell mediated killing of the cancer cells. In another aspect, the cancer drugs may inhibit the natural killer cell mediated killing of cancer cells, with or without affecting the viability of natural killer cells in patient. Such cancer drugs are administered before administering NK-92 cells, and the NK-92 cells are administered after the cancer drug is removed from the subject, or has been metabolized such that the level of drug is not inhibitory to NK-92

In another aspect, the cancer drugs are capable of stimulating natural killer cells to kill tumor cells. Such cancer drugs include, but are not limited to 5-fluorouracil, cyclophosphamide, paclitaxel, and gemcitabine.

### Methods of Treatment

In one aspect, the disclosure provides methods for treating cancer in a subject in need thereof. Cancers contemplated to be treated by the methods described herein include, for example, colorectal cancer, breast cancer, lung cancer, prostate cancer, pancreatic cancer, bladder cancer, cervical cancer, cholangiocarcinoma, leukemia, gastric sarcoma, glioma, lymphoma, melanoma, multiple myeloma, osteosarcoma, ovarian cancer, stomach cancer, brain cancer, and malignant mesothelioma. Treatment of both primary and metastatic cancers are contemplated.

In one aspect, the methods comprise administering a combination of an effective amount of cancer drugs (e.g., thalidamide , cisplatinuni, abraxane, or paclitaxel) and an effective amount of natural killer cells (e.g. NK-92 cells), which results in a synergistic effect for treating cancer. Specifically, administration of the cancer drug with natural killer cells will have a greater-than-additive effect in the treatment of cancer. For example, lower doses of one or more of the agents may be used in treating cancer, resulting in increased therapeutic efficacy and/or decreased side-effects.

In one aspect, the present disclosure provides methods of combination therapy for the treatment of cancer. Such combination therapy has several potential benefits. First, administration of a combination of a cancer drug and natural killer cells as described herein achieves a stronger cytotoxic effect on tumor cells than administration of either the cancer drug or the natural killer cells alone. Second, use of combination therapy reduces the necessary or minimum dose of both the cancer drug and the natural killer cells, thus lessening the morbidity associated with each. Third, the present disclosure contemplates the use of smaller doses of cancer drugs, which may decrease side-effects associated with use of such agents in subjects. Furthermore, the present disclosure allows the use of smaller doses of natural killer cells and cancer drugs, which decreases the cost of these expensive forms of anti-cancer therapy. The combined therapy described herein may be particularly useful for subjects who have inoperative and/or recurrent cancers which have proven resistant to conservative therapies.

In some embodiments, effective dosages and administration schedules of cancer drugs when used alone (i.e., administered in a composition without NK-92 cells) are those known in the art. Representative doses for cancer drugs (without combination therapy comprising NK-92 cells) are available in the Merck Manual Professional Edition (see the internet at merckmanuals.com/professional). Dosage, routes of administration, and administration schedules described in the art can be used, it being understood that the synergy demonstrated herein between such cancer drugs and natural killer cells allows the use of cancer drug at lower dosages than standard prior art dosages. For example, in some embodiments, dosage of cancer drugs from about 10% to 99% of prior art dosages can be used in combination therapy with NK-92 cells.

In some embodiments, the cancer drug is paclitaxel or its derivatives, and the effective dose when used in combination with NK cells is less than the standard dose for a given cancer. Representative adult human dosages, routes of administration, and administration schedules for paclitaxel are described in the Merck Manual (Id.) and include 80-225 mg/m² administered intravenously (IV) over a period of 3 to 24 hours, every 3 weeks, for breast cancer, non-small cell lung cancer, ovarian cancer, Kaposi sarcoma, bladder cancer, cervical cancer, head and neck cancers, and small cell lung cancer, soft tissue sarcoma (angiosarcoma), and thymoma/thymic carcinoma. The above representative dosages of paclitaxel can be combined with other chemotherapeutic agents depending on the type and stage of cancer, and the dose can vary based on the duration of the treatment cycle.

In some embodiments, the cancer drug is cisplatin or its derivatives, and the effective dose when used in combination with NK cells is less than the standard dose for a given cancer. Representative adult human dosages, routes of administration, and administration schedules for cisplatin are described in the Merck Manual (Id.) and include 50 to 100 mg/m² administered IV every 3 to 4 weeks for bladder cancer, ovarian cancer, cervical cancer, breast cancer (triple negative), endometrial carcinoma, head and neck cancer, non-small cell lung cancer, and small cell lung cancer; 100 mg/m² on days 1 and 29, or 60 mg/m² on day 1 every 3 weeks fur esophageal and gastric cancers; 10 mg/m²/day administered as a continuous infusion on days 1 to 4 of each cycle; repeat the cycle every 4 to 6 weeks for multiple myeloma (in combination with bortezomib, dexamethasone, thalidomide, doxorubicin, cyclophosphamide, and etoposide); 25 mg/m²/day administered as a continuous infusion over a 24 hour period on days 1 to 4, repeat every 3 to 4 weeks for 6 to 8 cycles for Non-Hodgkin lymphoma (in combination with etoposide, methylprednisolone, and cytarabine, ESHAP regimen); and 25 mg/m² administered over 2 hours on days 1, 2, and 3, repeat every 3 to 4 weeks (in combination with paclitaxel and ifosfamide) for 4 cycles for penile cancer. The above representative dosages of cisplatin can be combined with other chemotherapeutic agents depending on the type and stage of cancer, and the dose can vary based on the duration of the treatment cycle.

In some embodiments, the cancer drug is thalidomide or its derivatives, and the effective dose when used in combination with NK cells is less than the standard dose for a given cancer. Representative adult human dosages, routes of administration, and administration schedules for thalidomide are described in the Merck Manual (Id.) and include 100 to 200 mg administered orally once daily over various treatment cycles for multiple myeloma. The treatment cycles include, e.g., 100 mg once daily for the first 14 days, then 200 mg once daily for three, 21-day cycles, or 100 mg per day for up to eight, 21-day cycles, or 100-200 mg per day starting 42 days to 6 months after transplant and continuing until disease progression, or up to 12 months. The above representative dosages of thalidomide can be combined with other chemotherapeutic agents or steroids, and the dose can vary based on the duration of the treatment cycle.

It will be understood that the effective dose of a single cancer drug, when used in combination with NK cells, can be less than the standard dose for a given cancer due to the synergistic effect when combined with NK-92 cells, Likewise, the effective dose for each drug in a combination of cancer drugs, such as paclitaxel and cisplatin, can be less than the standard dose for each drug for a given cancer due to the synergistic effect when combined with NK-92 cells.

In some embodiments, the dose of the cancer drug is a metronomic dose, i.e., a low, continuous dose. Previous studies show that metronomic chemotherapy can be more effective than high-dose therapy in patients with advanced breast cancer (see, e.g., Montagna E, Cancello G, Dellapasqua S, Munzone E, Colleoni M, Cancer Treat Rev. 2014; 40(8):942-950).

Further, doses of cancer drugs administered to animals can be converted to equivalent doses for humans based on the body surface area (BSA) (represented in mg/m²) normalization method (see, e.g., Reagan-Shaw, S. et al., “Dose translation from animal to human studies revisited,” FASEB J. 22, 659-661 (2007); and “Guidance for Industry—Estimating the Maximum Safe Starting Dose in Initial Clinical Trials for Therapeutics in Adult Healthy Volunteers,” U.S. Department of Health and Human Services, Food and Drug Administration, Center for Drug Evaluation and Research (CDER), July 2005, Pharmacology and Toxicology; which are incorporated by reference herein). For example, the human equivalent dose (HED) based on BSA is can be calculated by the following formula I:

HED animal dose in mg/kg×animal weight in kg/human weight in kg)^{0.33} I.

Alternatively, the HED can be determined by the following formula II:

HED (mg/kg)=animal dose g/kg)×(animal Kₘ/human Kₘ) II.

The Km factor is determined based on the following Table (see Guidance for Industry, Id.):

TABLE 1
Conversion of Animal Doses to Human Equivalent
Doses Based on Body Surface Area

| | To Convert | To Convert Animal Dose in mg/kg | |
|---|---|---|---|
| | Animal Dose in | to HED^{a} in mg/kg, Either: | |
| | mg/kg to Dose in | Divide | Multiply |
| | mg/m², Multiply | Animal Dose | Animal Dose |
| Species | by kₘ | By [symbol] | By [symbol] |
| Human | 37 | — | — |
| Child (20 kg)^{b} | 25 | — | — |
| Mouse | 3 | 12.3 | 0.08 |
| Hamster | 5 | 7.4 | 0.13 |
| Rat | 6 | 6.2 | 0.16 |
| Ferret | 7 | 5.3 | 0.19 |
| Guinea pig | 8 | 4.6 | 0.22 |
| Rabbit | 12 | 3.1 | 0.32 |
| Dog | 20 | 1.8 | 0.54 |
| Primates: | | | |
| Monkeys^{c} | 12 | 3.1 | 0.32 |
| Marmoset | 6 | 6.2 | 0.16 |
| Squirrel monkey | 7 | 5.3 | 0.19 |
| Baboon | 20 | 1.8 | 0.54 |
| Micro-pig | 27 | 1.4 | 0.73 |
| Mini-pig | 35 | 1.1 | 0.95 |

Assumes 60 kg human. [symbol]
indicates data missing or illegible when filed

Thus, a 5 mg/kg dose in mice is equivalent to a 0.4 mg/kg dose in a 60 kg human. A 0.4 mg/ml dose in a 60 kg human is equivalent to a dose of 14.8 mg/m².

In some embodiments, the compositions described herein are administered in therapeutically effective amounts for periods of time effective to treat a cancer or tumor. The effective amount of the cancer drugs in combination with the NK-92 cells described herein can be determined by one of ordinary skill in the art and includes exemplary dosage amounts for a mammal of from about 0.5 to about 200 mg/kg, about 0.5 to about 150 mg/kg, about 0.5 to 100 mg/kg, about 0,5 to about 75mg/kg, about 0.5 to about 50mg/kg, about 0.01 to about 50mg/kg, about 0.05 to about 25 mg/kg, about 0.1 to about 25 mg/kg, about 0.5 to about 25 mg/kg, about 1 to about 20 mg/kg, about 1 to about 10 mg/kg, about 20mg/kg of body weight, about 10 mg/kg, about 5 mg/kg, about 2.5 mg/kg, about 1.0 mg/kg, or about 0.5 mg/kg of body weight of the cancer drug, or any range derivable therein. In some embodiments, the dosage amounts of the cancer drug are from about 0.01 mg/kg to about 10 mg/kg of body weight. In some embodiments, the dosage amount of the cancer drug is from about 0.01 mg/kg to about 5 mg/kg, or from about 0.01 mg/kg to about 2.5 mg/kg of body weight. The compositions described can be administered in a single dose or in the form of individual divided doses, such as from 1 to 4 times per clay, or once every 2 days, 3 days, 4 days, 5 days, 6 days, weekly, or monthly. The compositions described herein can also be administered for various treatment cycles, such as 2, 3, 4, 5, 6, 7, 8, 9, 10 treatment cycles. The treatment cycles can be different lengths of time depending on the cancer to be treated, for example, 1, 2, 3, 4, 5, 6, 7, 8, 9, or 10 week treatment cycles.

Similarly, for natural killer cells, dosages, routes of administration, and administration schedules described in prior art may be used, again with the understanding that the synergy demonstrated herein between such cancer drugs and natural killer cells allows the use of cancer drug at lower dosages lower than standard prior art dosages. For example, in addition to prior art dosages, dosage of cancer drugs from about 10% to 99% of prior art dosages can be used.

The effective amount can be determined during pre-clinical trials and clinical trials by methods familiar to physicians and clinicians. An effective amount of a peptide useful in the methods can be administered to a subject in need thereof by any of a number of well-known methods for administering pharmaceutical compounds.

The natural killer cells and the cancer drugs can be administered by any suitable delivery route and may include, without limitation, parenteral, subcutaneous, subdural, intramuscular, intrathecal, or intraperitoneal injection.

In certain embodiments, the compositions comprise at least one additive such as a filler, bulking agent, buffer, stabilizer, or excipient. Standard pharmaceutical formulation techniques are well known to persons skilled in the art (see, e.g., 2005 Physicians' Desk Reference©, Thomson Healthcare: Montvale, N.J., 2004; Remington: The Science and Practice of Pharmacy, 20th ed., Gennado et al., Eds. Lippincott Williams & Wilkins: Philadelphia, Pa., 2000). Suitable pharmaceutical additives include, e.g., mannitol, starch, glucose, lactose, sucrose, gelatin, silica gel, sodium stearate, glycerol monostearate, talc, sodium chloride, glycerol, propylene, glycol, water, ethanol, and the like. In certain embodiments, the pharmaceutical compositions contain pH buffering reagents and wetting or emulsifying agents. In further embodiments, the compositions may contain preservatives or stabilizers.

The formulation of the compositions can vary depending on the intended route of administrations and other parameters (see, e.g., Rowe et al., Handbook of Pharmaceutical Excipients, 4th ed. APhA Publications, 2003.) In some embodiments, the composition is a lyophilized cake or powder. The lyophilized composition can be reconstituted for administration by intravenous injection, for example with Sterile Water for Injection, USP. In other embodiments, the composition is a sterile, non-pyrogenic solution. In still further embodiments, the composition is delivered in powder form in a pill or tablet.

The NK-92 cells and the cancer drugs can be administered separately or admixed and administered together. In certain embodiments, the NK-92 cells and the cancer drugs are administered together in a single intravenous administration. In other embodiments, the NK-92 cells are administered first, followed by administration of the cancer drugs. In other embodiments, the cancer drugs are administered first, followed by administration of the NK-92 cells. In certain embodiments, the timing between the administrations may be approximately 5, 10, 20, 30, 40, 50, 60, 90, or 120 minutes. In other embodiments, the timing between the administrations may vary from zero to 24 hours, from 1 day to 7 days, from more than zero weeks to less than four weeks. In some embodiments, the cancer drugs or the NK-92 cells are given in multiple doses, or both can be given as multiple doses.

In some embodiments, the combination therapy of NK-92 and cancer drugs described herein applies to acute therapy of cancer that is designed to treat existing cancers or decrease the size of bulk tumors. Thus, in some embodiments, the combination therapy is not designed for so-called “consolidation therapy” to eliminate or reduce the number of cancer stem cells or residual tumor cells after a patient is treated with conventional chemotherapy. In some embodiments, the combination therapy of NK-92 and cancer drugs described herein can be used to treat a patient whose cancer has relapsed from previous treatment, either after conventional chemotherapy or by combination therapies described herein.

In certain embodiments, treatment of cancer using a combination of NK-92 cells and cancer drugs potentiates the therapeutic effect of either NK-92 cells or cancer drugs in isolation. In some embodiments, combination therapy results in an additive effect on the treatment of cancer. In further embodiments, the combination acts synergistically to enhance the effect seen with either NK-92 cells or cancer drugs in isolation.

In certain embodiments of the combination therapy, NK-92 cells and cancer drugs are administered at reduced dosages, as compared to the optimal concentration when administered individually. In these embodiments, the combination therapy enables the use of reduced concentrations while maintaining therapeutic or other beneficial effects, thereby reducing cost and the risk of adverse reaction to the therapeutic agents. In certain embodiments, the use of reduced dosages could also lead to increased subject tolerance.

Administration to a subject may occur in a single dose or in repeat administrations, and in any of a variety of physiologically acceptable forms, and/or with an acceptable pharmaceutical carrier and/or additive as part of a pharmaceutical composition.

The composition comprising NK-92 cells and the composition comprising cancer drugs or the composition comprising both NK-92 and cancer drugs may be administered to a subject in effective amounts. Generally, an effective amount may vary with the subject's age, general condition, and gender, as well as the severity of the medical condition in the subject. The dosage may be determined by a physician and adjusted, as necessary, to suit the observed effects of the treatment.

In certain embodiments, the NK-92 cells are administered to a subject by absolute numbers of cells, e.g., said subject is administered from about 1000 cells/injection to up to about 10 billion cells/injection, such as about, at least about, or at most about, 1×10⁸, 1×10⁷, 5×10⁷, 1×10⁶, 5×10⁶, 1×10⁵, 5×10⁵, 1×10⁴, 5×10⁴, 1×10³, 5×10³ (and so forth) NK-92 cells per injection, or any ranges between any two of the numbers, end points inclusive. In other embodiments, NK-92 cells are administered to an individual by relative numbers of cells, e.g., said individual is administered about 1000 cells to up to about 10 billion cells per kilogram of the individual, such as at about, at least about, or at most about, 1×10⁸, 1×10⁷, 5×10⁷, 1×10⁶, 5×10⁶, 1×10⁵, 5×10⁵, 1×10⁴, 5×10⁴, 1×10³, 5×10³ (and so forth) NK-92 cells per kilogram of the individual, or any ranges between any two of the numbers, end points inclusive. In other embodiments, the total dose may calculated by m² of body surface area, including 1×10¹¹, 1×10¹⁰, 1×10⁹, 1×10⁸, or 1×10⁷ per m². The surface area of the average human is 1.6-1.8 m².

In some embodiments of the method, said treatment for cancer includes a conventional therapy, such as chemotherapy in combination with a composition comprising at least one modified NK-92 cell and an amount of cancer drugs.

In a further embodiment, the compositions described herein are administered to a subject in conjunction with (e.g., before, simultaneously or following) conventional therapies, such as chemotherapy. For example, in one embodiment, subjects may undergo standard treatment with high dose chemotherapy followed by an infusion of NK-92 cells described herein.

### EXAMPLES

The following examples are for illustrative purposes only and should not be interpreted as limitations of the claimed invention. There are a variety of alternative techniques and procedures available to those of skill in the art which would similarly permit one of ordinary skill in the art to successfully perform the claimed invention.

### Example 1

### Combination Therapy Using Cancer Drugs and NK-92 Cells to Treat Cancer

This example demonstrates that combining low dose paclitaxel treatment with a HER2-specific NK-92 cell line resulted in synergistic reduction in tumor growth in vivo.

Methods: HER2.taNK cells, a stable clonal HER2-specific NK-92 cell line that mediated selective and sequential killing of HER2-expressing MDA-MB-453 cells in vitro, were generated as described previously (Schonfeld K, Sahni C, Zhang C, et al. Mol Ther. 2015; 23(2):330-338). Nant-paclitaxel is a lyophilized polymeric micellar formulation of paclitaxel that is approved outside the United States for a number of cancer indications. aNK cells are unmodified, activated NK-92 cells that showed no evidence of cytokine storm from 18 infusions delivered over 6 months; clinical responses were observed in a subset of patients (see, Arai S, Meagher R, Swearingen M, et al. Cytotherapy. 2008; 10(6):625-632; Tonn T, Schwabe D, Klingemann H G, et al. Cytotherapy. 2013; 15(12):1563-1570).

Metronomic (low-dose, continuous) chemotherapy can be more effective than high-dose therapy in patients with advanced breast cancer (Montagna E, Cancello G, Dellapasqua S, Munzone E, Colleoni M, Cancer Treat Rev. 2014; 40(8):942-950).

MDA-MB-453 cells (0.1 mL of 1×108 cells/mL in 50% Matrigel) were injected SC into the left and right flank area of female NOD/SCID mice (7 to 8 weeks old). When tumors reached ˜100 mm3, mice were randomly assigned to 4 groups of 4 mice/group and dosed (IV) with saline, nant-paclitaxel, γ-irradiated (10 Gy) aNK cells/HER2.taNK cells, or nant-paclitaxel+γ-irradiated (10 Gy) aNK cells/HER2.taNK cells. γ-irradiation prevents aNK/HER2.taNK cell replication.

Tumor growth was measured with calipers twice weekly prior to dosing, then twice weekly; animals were weighed before injection of cells, before dosing, then twice weekly. Data are presented as means±SEM. Statistical analysis was done using ANOVA and Student's t-test.

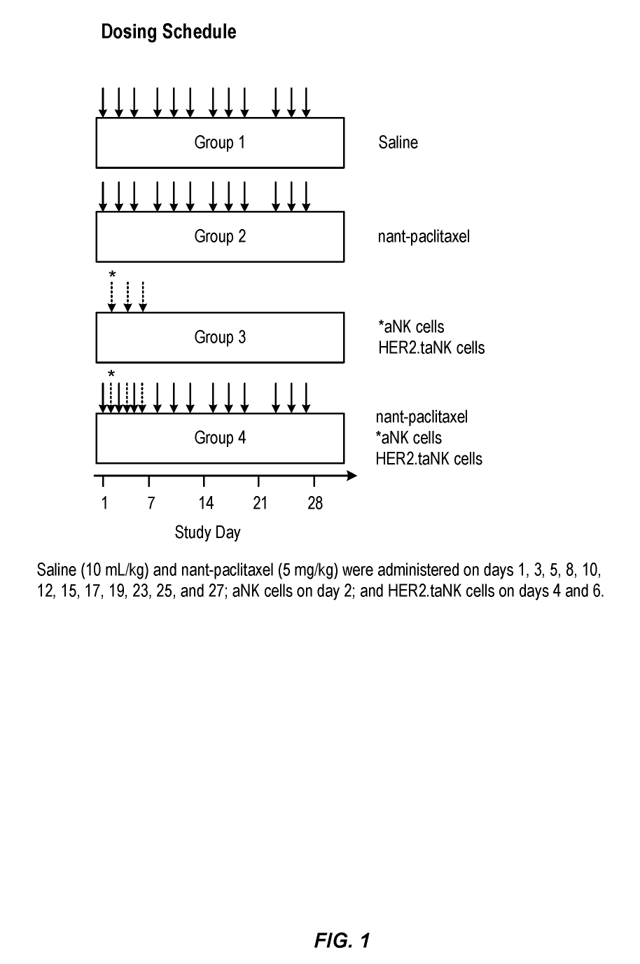

FIG. 1 shows the dosing schedule.

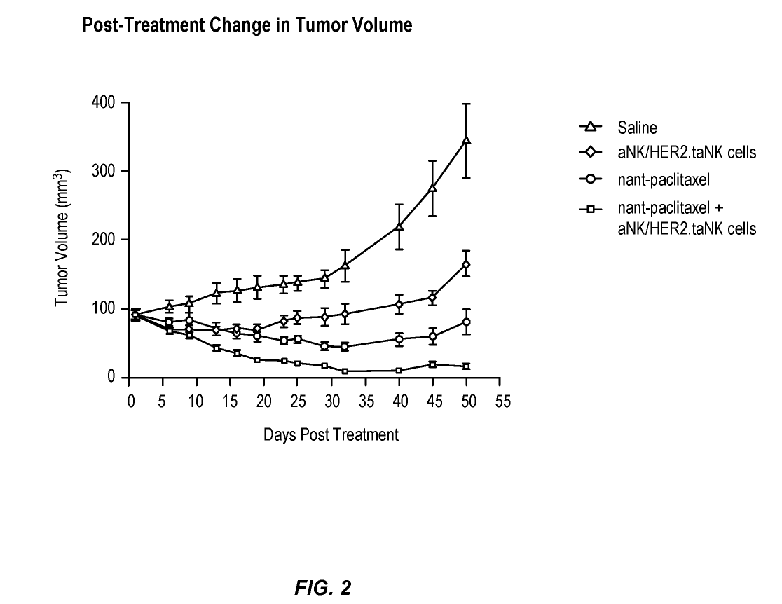

FIG. 2 shows post-treatment change in tumor volume.

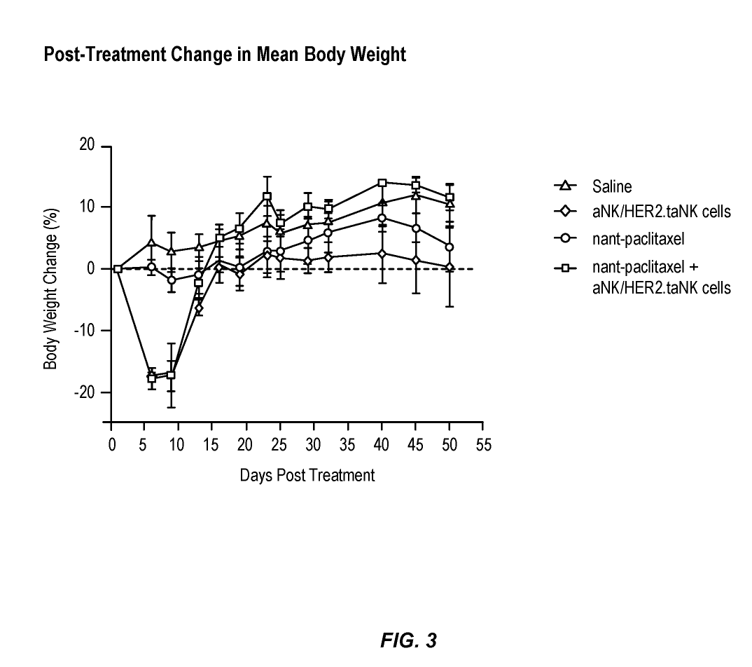

FIG. 3 shows the post-treatment change in mean body weight.

Table 2 shows the amount of Tumor Growth Inhibition at Day 32.

TABLE 2

| | | T/C | |
|---|---|---|---|
| Treatment | Dose | (%) | P-value |
| Nant-paclitaxel | 5 mg/kg | −48.6 | P < 0.0007 (vs saline) |
| aNK/HER2.taNK | 1 × 107 cells | 0 | P < 0.006 (vs saline) |
| Nant-paclitaxel + | 5 mg/kg + 1 × 10⁷ | −89.6 | P < 0.003 (vs nant- |
| aNK/HER2.taNK | cells | | paclitaxel); |
| | | | P < 0.0002 (vs. aNK/ |
| | | | HER2.taNK) |

T/C = tumor growth inhibition ratio.

### RESULTS AND CONCLUSIONS

Nant-paclitaxel alone and aNK cells/HER2.taNK cells alone significantly inhibited tumor growth in this mouse model of HER2-positive breast cancer. The combination of nant-paclitaxel plus aNK cells/HER2.taNK cells appeared to be synergistic resulting in significant tumor regressions and significantly better efficacy vs. each agent alone HER2.taNK cells were administered only twice early in the study, yet imparted a lasting impact on tumor growth. While not being bound by theory, a potential mechanism for the synergy between low-dose paclitaxel and NK cell-based immunotherapy demonstrated in this study is paclitaxel-induced immunostimulation of tumors for increased recognition and killing by the tumor-targeted NK-92 platform. The results of this study demonstrate that combining metronomic (low-dose) chemotherapy with NK-based immunotherapy is more effective than either treatment alone in a well-accepted in vivo model for breast cancer, and suggest similar treatments may be effective in human patients with metastatic breast cancer.

### Example 2

Suitable in vitro or in vivo assays can be performed to determine the effect of the combination therapy. Compounds for use in therapy can be tested in suitable animal model systems including, but not limited to bovines, swine, rabbits, alpacas, horses, canines, felines, ferrets, rats, mice, fowl and buffalo and the like, prior to testing in human subjects. Similarly, for in vivo testing, any well-accepted animal model system known in the art can be used prior to administration to human subjects. For in vitro testing, any cancer cell line model systems known in the art can be used.

Based on the results presented in Example 1, it is expected that the combination of NK-92 cells and the cancer drugs will show a synergistic effect of killing or inhibiting tumor growth. Specifically, The combination of NK-92 cells and cancer drug is expected to potentiate the therapeutic effect of either NK-92 cells or cancer drugs in isolation. It is also expected that the combination of NK-92 cells and the cancer drug enables the use of reduced concentrations while maintain therapeutic or other beneficial effects. In certain experiments, it is also expected that the reduced dosages could lead to increased subject tolerance.

It is understood that the examples and embodiments described herein are for illustrative purposes only and that various modifications or changes in light thereof will be suggested to persons skilled in the art and are to be included within the spirit and purview of this application and scope of the appended claims. All publications, sequence accession numbers, patents, and patent applications cited herein are hereby incorporated by reference in their entirety for all purposes.

1.-27. (canceled)

28. A method of treating a subject having a cancer, comprising administering to the subject an effective amount of at least one cancer drug and at least one NK-92 cell.

29. The method of claim 28, wherein the cancer is selected from the group consisting of colorectal cancer, breast cancer, lung cancer, prostate cancer, pancreatic cancer, bladder cancer, cervical cancer, cholangiocarcinoma, gastric sarcoma, glioma, leukemia, lymphoma, melanoma, multiple myeloma, osteosarcoma, ovarian cancer, stomach cancer, brain cancer.

30. The method of claim 28, wherein the cancer drug is selected from the group consisting of: thalidomide, cisplatin (cis-DDP), oxaliplatin, carboplatin, anthracenediones, mitoxantrone; hydroxyurea, methylhydrazine derivatives, procarbazine (N-methylhydrazine, MIH), adrenocortical suppressants, mitotane (o,p′-DDD), aminoglutethimide, RXR agonists, bexarotene, tyrosine kinase inhibitors, sunitinib, imatinib, mechlorethamine, cyclophosphamide, ifosfamide, melphalan (L-sarcolysin), chlorambucil, ethylenimines, methylmelamines, hexamethylmelamine, thiotepa, busulfan, carmustine (BCNU), semustine (methyl-CCNU), lomustine (CCNU), streptozocin (streptozotocin), DNA synthesis antagonists, estramustine phosphate, triazines, dacarbazine (DTIC, dimethyl-triazenoimidazolecarboxamide), temozolomide, folic acid analogs, methotrexate (amethopterin), pyrimidine analogs, fluorouracin (5-fluorouracil, 5-FU, 5FU), floxuridine (fluorodeoxyuridine, FUdR), cytarabine (cytosine arabinoside), gemcitabine, purine analogs, mercaptopurine (6-mercaptopurine, 6-MP), thioguanine (6-thioguanine, TG), pentostatin (2′-deoxycoformycin, deoxycoformycin), cladribine and fludarabine, topoisomerase inhibitors, amsacrine, vinca alkaloids, vinblastine (VLB), vincristine, taxanes, paclitaxel, protein bound paclitaxel (Abraxane), Nant-paclitaxel, docetaxel (Taxotere); epipodophyllotoxins, etoposide, teniposide, camptothecins, topotecan, irinotecan, dactinomycin (actinomycin D), daunorubicin (daunomycin, rubidomycin), doxorubicin, bleomycin, mitomycin (mitomycin C), idarubicin, epirubicin, buserelin, adrenocorticosteroids, prednisone, progestins , hydroxyprogesterone caproate, medroxyprogesterone acetate, megestrol acetate, diethylstilbestrol, ethinyl estradiol, tamoxifen, anastrozole; testosterone propionate, fluoxymesterone, flutamide, bicalutamide, bortezomib, and leuprolide.

31. The method of claim 28, wherein the cancer drug is thalidomide or its derivatives.

32. The method of claim 28, wherein the cancer drug is selected from the group consisting of cisplatin, carboplatin, and oxaliplatin.

33. The method of claim 28, wherein the cancer drug is selected from the group consisting of paclitaxel, Nant-paclitaxel, Abraxane, and Taxotere.

34. The method of claim 28, wherein the cancer drug is selected from the group consisting of asparaginase, bevacizumab, bleomycin, doxorubicin, epirubicin, etoposide, 5-fluorouracil, hydroxyurea, streptozocin, and 6-mercaptopurine, cyclophosphamide, and gemcitabine.

35. The method of claim 28, wherein the cancer drug and the NK-92 cells are administered simultaneously.

36. The method of claim 28, wherein the cancer drug and the NK-92 cells are administered sequentially.

37. The method of claim 28, wherein the cancer drug and the NK-92 cells are mixed together prior to administering to the subject.

38. The method of claim 28, wherein the cancer drug is administered before the administration of the NK-92 cells, and wherein the NK-92 cells are administered after the cancer drug is removed from the subject.

39. The method of claim 28, wherein the effective amount of the cancer drug administered to the subject is less than the effective amount of the drug administered alone.

40. The method of claim 28, wherein the combination of at least one cancer drug and at least one NK-92 cell provides a synergistic result compared to the administration of the cancer drug or NK-92 cell alone.

41. The method of claim 40, wherein the cancer drug is paclitaxel, and the cancer is breast cancer.

42. The method of claim 28, wherein the NK92 cell is modified to express at least one marker on the surface of the cell.

43. The method of claim 42, wherein the at least one marker is an Fc-gamma receptor or CD16.

44. The method of claim 28, wherein the NK92 cell is modified to express a chimeric antigen receptor (CAR) that binds to an antigen on a cancer or tumor cell.

45. The method of claim 44, wherein the CAR binds to the ErbB2 (HER2) antigen.

46. A composition comprising at least one NK-92 cell and at least one cancer drug.
